(19) United States
(12) Patent Application Publication
(10) Pub. No.: US 2024/0197475 A1
(43) Pub. Date: Jun. 20, 2024

(54) IMPLANT FOR HEART VALVE REPAIR

(71) Applicants: UNIVERSITE TECHNOLOGIE DE COMPIEGNE, COMPIEGNE (FR); CENTRE NATIONAL DE LA RECHERCHE SCIENTIFIQUE (CNRS), PARIS (FR); ASSISTANCE PUBLIQUE-HOPITAUX DE PARIS, PARIS (FR); FONDAZIONE RI. MED, PALERMO (IT)

(72) Inventors: Anne-Virginie SALSAC, SAINT JEAN AUX BOIS (FR); Gaëtano BURRIESCI, LONDON (GB); Antonio FIORE, CHARENTON LE PONT (FR)

(21) Appl. No.: 18/288,584

(22) PCT Filed: Apr. 28, 2021
(86) PCT No.: PCT/IB2021/000316

§ 371 Date: Oct. 27, 2023

(51) Int. Cl. A61F 2/24

(52) U.S. Cl. CPC A61F 2/246; A61F 2220/0016

(57) ABSTRACT

An implant for heart valve repair, the implant comprising: a clamp movable between an open position and a closed position in which the clamp is able to grasp a leaflet of a heart valve, so as to fix the implant on the leaflet; and a shutter comprising a structure adapted to pass automatically from a contracted configuration to an expanded configuration in which the shutter is able to fill at least partially an opening portion remaining between the leaflet and at least one further leaflet of the heart valve of the heart valve, when the implant is attached to one leaflet, so as to limit a backflow of blood through the opening portion when the heart valve closes, and wherein the structure is curved in two directions perpendicular to each other in the expanded configuration.

### TECHNICAL FIELD

The present disclosure relates to a cardiac implant for the treatment of valve leakage. It is particularly suitable for the treatment of mitral insufficiency in a patient, but its use may be extended to other heart valves in addition to the mitral valve.

### BACKGROUND

FIG. 1 shows a patient's heart. It comprises a left ventricle LV, separated from the left atrium LA by the mitral valve MV. When the left ventricle LV contracts (systole), the left atrium LA fills with oxygenated blood from the pulmonary veins PV and the mitral valve MV is closed. The left ventricle LV then relaxes (diastole). The mitral valve MV opens and blood flows from the left atrium LA to the left ventricle LV. The left ventricle then contracts, closing the mitral valve MV and opening the aortic valve AV, in order to eject the oxygenated blood into the aorta AO, towards the rest of the body.

As shown in FIGS. 2a and 2b, the mitral valve MV consists of a mitral annulus MA, which delimits the opening between the left atrium LA and the left ventricle LV, a posterior leaflet PL and an anterior leaflet AL. The posterior leaflet PL and the anterior leaflet AL extend from the posterior and the anterior parts of the mitral annulus MA, respectively. The Sub-Valvular Mitral Appliance, SVMA, consists of tendon chords (chordae tendineae) connecting the margins of the posterior and anterior leaflets, PL and AL, to the wall of the left ventricle LV via the papillary muscles.

FIGS. 2a and 2b show, in top view, a healthy mitral valve MV in diastole and systole, respectively. During diastole, the anterior and posterior leaflets AL and PL of the mitral valve MV diverge from each other, allowing blood to flow from the left atrium LA to the left ventricle LV. During systole, the anterior and posterior leaflets AL and PL of the mitral valve MV come into contact with each other so as to close the gap between the left ventricle LV and the left atrium LA, thus ensuring coaptation of the leaflets and preventing blood from flowing back from the left ventricle LV into the left atrium LA.

FIGS. 3a and 3b show, in top view, a mitral valve MV of a patient with mitral insufficiency, during diastole (relaxation movement of the heart) and systole (contraction movement of the heart), respectively. It can be seen in FIG. 3b that part of the posterior leaflet PL is unable to come into contact with the anterior leaflet AL during systole. The mitral valve MV does not close properly, causing regurgitation—reflux—of blood from the left ventricle LV into the left atrium LA. This reduces the efficiency of the heart cycle, potentially leading to heart failure.

This is called mitral insufficiency. In the case of secondary or functional insufficiency (when it is not due to defects of the constituents of the valve itself but to anomalies of the subvalvular apparati), the leakage is often caused by the dilatation of the left ventricle LV. This dilatation leads to an increase in the diameter of the mitral annulus MA, which causes an even larger gap between the posterior leaflet PL and anterior leaflet AL during systole, and thus further increases the mitral insufficiency (thus creating a vicious circle).

Apart from pharmacological treatment, which is mostly palliative, the standard treatment for this condition consists in valve repair or surgical replacement. When this pathology is secondary to cardiomyopathy (secondary or functional mitral insufficiency), surgical intervention presents a significant risk, due to comorbidities or the advanced age of the patient.

Several percutaneous valve repair solutions to treat mitral insufficiency and limit the invasive nature of the treatment have therefore been proposed such as leaflets plication procedures, chordae repair and direct or indirect annuloplasty solutions. However, current approaches have been demonstrated to be suitable only for very specific forms of mitral valve disease and anatomic subset, and do not offer a degree of efficacy equivalent to surgical repair.

An approach of more general applicability was proposed in FR1858233, the implant comprising jaws to attach the implant to a first leaflet of the heart valve, and an inflatable balloon to occlude at least a portion of the opening remaining in the leaflet in the event of mitral insufficiency. Contrary to other devices, this implant is not attached to the second leaflet of the heart valve, which is therefore free and can work as usual.

However, repeated contacts of the balloon with the second leaflet could damage the balloon. Moreover, the use of a balloon requires filling the balloon with a fluid. The volume of the balloon must therefore be adjusted either before or during the operation, which complicates the implantation procedure by lengthening the duration of the operation.

### SUMMARY

The present disclosure aims at offering a heart valve implant that provides effective repair, can be easily implanted, is adapted to the severity of mitral insufficiency, and has a longer lifespan than a conventional valve.

To that end, the present disclosure proposes an implant for heart valve repair, the implant comprising:
a clamp movable between an open position and a closed position in which the clamp is able to grasp a leaflet of a heart valve, so as to fix the implant on the leaflet, and
a shutter comprising a structure adapted to pass automatically from a contracted configuration to an expanded configuration in which the shutter is able to fill at least partially an opening portion remaining between the leaflet and at least one further leaflet of the heart valve of the heart valve, when the implant is attached to one leaflet, so as to limit a backflow of blood through the opening portion when the heart valve closes, and wherein the structure is curved in two directions perpendicular to each other in the expanded configuration.

This implant alleviates the aforementioned disadvantages, while retaining its main function of repairing the mitral valve. The implant thus created will be able to fold up in order to be easily inserted into a catheter, then to unfold so as to ensure its function as an implant for the heart valve. This device makes it possible to keep a leaflet of the heart valve on which the implant is not fixed completely free and mobile, so that it maintains normal physiological function, especially during systole. The contact between the implant and the other leaflet is facilitated, ensuring a good seal of the heart valve.

The implant may further comprise the optional feature set forth below, taken alone or combined whenever possible.

The structure may be a metallic mesh.

The structure may be made of a polymer selected from the group consisting of polyolefin, vinyl polymer, saturated polyester, polyurethane or any other biocompatible polymer.

The shutter may comprise a membrane at least partially covering the structure.

In an embodiment, the shutter defines a first bearing surface, and the clamp comprises a fastening arm defining a second bearing surface capable of facing the first bearing surface when the clamp is in the closed position, such that the first bearing surface and the second bearing surface enclose the leaflet.

At least one of the first bearing surface and the second bearing surface may be serrated or corrugated.

The fastening arm may be rotatable or bendable about an axis of rotation relative to the structure, and may have a zig zag shape.

In an embodiment, the clamp comprises at least one further fastening arm defining a third bearing surface capable of facing the first bearing surface when the clamp is in the closed position, such that the first bearing surface, the second bearing surface and the third bearing surface enclose the first leaflet therebetween.

The shutter may comprise wires which are free to move relative to each other when the meshed structure passes from the contracted configuration to the expanded configuration, the implant further comprising a sleeve enclosing end portions of the wires.

In an embodiment, the clamp comprises a fastening arm, and the sleeve encloses an end portion the fastening arm.

The wires and the fastening arm may protrude out of the sleeve through an opening of the sleeve. Alternatively, the sleeve may have a first opening and a second opening opposite the first opening, the wires protrude out of the sleeve through the first opening and the fastening arm protrudes out of the sleeve through the second opening.

The structure and the clamp may be parts of a single piece.

### BRIEF DESCRIPTION OF THE FIGURES

Further details, features and advantages are explained in more detail below with the aid of exemplary embodiments that are illustrated in the figures.

FIG. 1 schematically shows a patient's heart with the positioning of the mitral valve et of three other cardiac valves;

FIGS. 2a and 2b schematically show, in top view, a healthy mitral valve in diastole and systole, respectively,

FIGS. 3a and 3b schematically show, in top view, a mitral valve of a patient with mitral insufficiency, during diastole and systole, respectively,

FIG. 4 schematically shows an implant for a mitral valve of a patient suffering from mitral insufficiency according to a first embodiment of the invention,

FIGS. 5a to 5d schematically illustrate a clamp of the first implant according to the first embodiment,

FIG. 6a schematically show a shutter of the first implant according to the first embodiment,

FIG. 6b schematically illustrate the first implant according to a first embodiment in a catheter,

FIGS. 7a to 7c schematically show a second shutter,

FIGS. 8a to 8c schematically show a third shutter,

FIGS. 9a to 9c schematically show a fourth shutter,

FIGS. 10a to 10k schematically illustrates steps of a method of placing an implant by a transseptal route,

FIGS. 11a and 11b schematically show a second implant according to a second embodiment of the invention,

FIG. 12 schematically shows a third implant according to a third embodiment of the invention,

FIG. 13 schematically shows a variant of the second implant with two fastening arms,

FIG. 14 schematically shows another variant of the second implant with a serrated or corrugated fastening arm,

FIG. 15 schematically show a fourth implant according to a fourth embodiment,

FIGS. 16a and 16b schematically show a fifth implant according to a fifth embodiment of the invention.

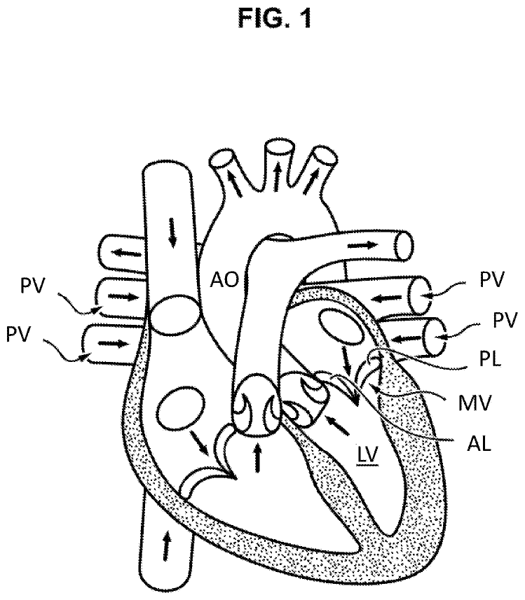

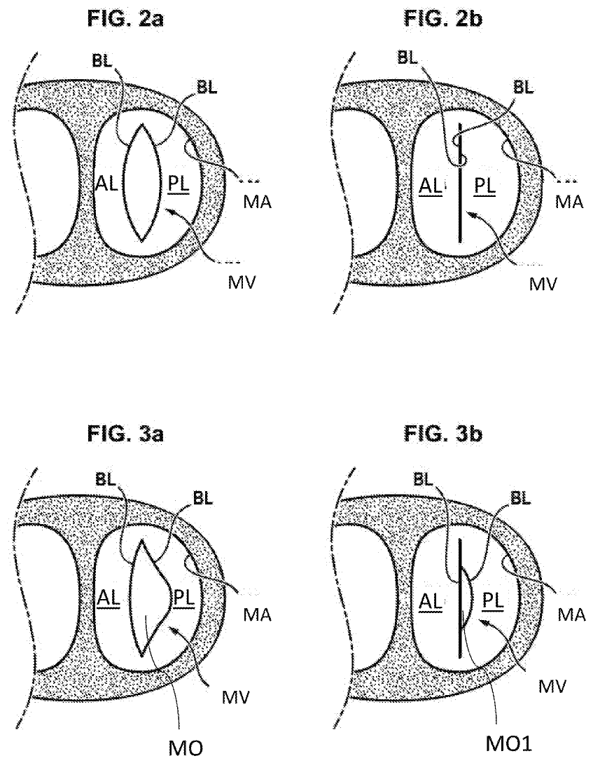

### DETAILED DESCRIPTION OF EMBODIMENTS

As described above, FIGS. 3a and 3b show the mitral valve MV of a patient with mitral insufficiency, during diastole and systole, respectively. During diastole, the anterior and posterior leaflets AL and PL of the mitral valve MV delimit between them a mitral orifice MO which allows blood to flow from the left atrium LA to the left ventricle LV. The mitral valve MV is then open. During systole, the anterior and posterior leaflets AL and PL of the mitral valve MV only partially come into contact with each other, leaving a gap MO1. The mitral valve MV is therefore only partially closed, causing a reflux of blood from the left ventricle LV to the left atrium LA.

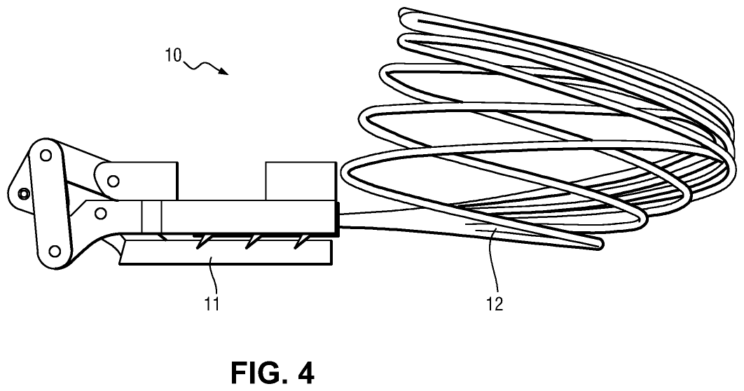

FIG. 4 schematically shows an implant 10 for a mitral valve MV of a patient suffering from mitral insufficiency according to a first embodiment.

The implant 10 comprises a clamp 11 movable between an open position and a closed position in which the clasp is able to clamp a first leaflet among leaflets AL, PL of the mitral valve MV, so as to fix the implant on the first leaflet.

In the first embodiment, the first leaflet is clamped between two bearing surfaces of the implant 10: a first bearing surface of the body 13, and a second bearing surface of the jaw 14. Preferably, the implant is clamped to the posterior leaflet PL, as the stability of the implant is likely to be better than if it is attached to the anterior leaflet AL. It is also more indicated to place it on the posterior leaflet PL, which is larger, less mobile and more prone to calcification than the anterior leaflet AL.

The implant 10 further comprises a shutter 12, which will be described later.

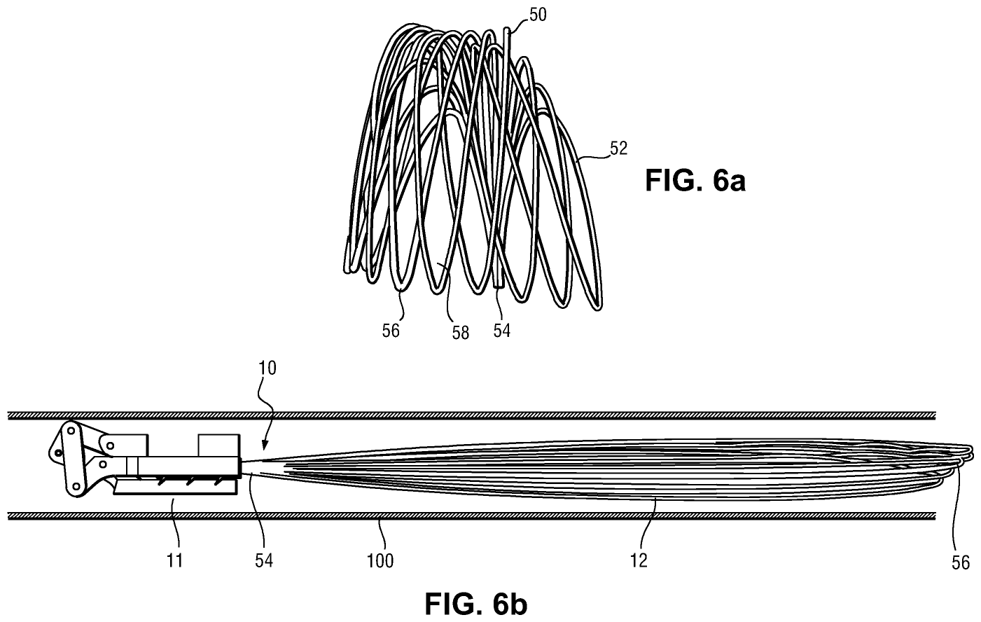

Implant 10 is designed for insertion into a main catheter 100 (shown in FIG. 6b), so that it can be implanted percutaneously or transatrially. When implant 10 is percutaneously implanted, for example, the main catheter is inserted into the femoral vein at the groin fold and is threaded transeptally (through the interatrial septum, the membrane that separates the left atrium LA from the right atrium RA) up to the left atrium LA and then up to the mitral valve MV. Implantation is possible through the femoral route when the diameter of the catheter containing the implant is sufficiently small, typically less than 8 mm in diameter, or through an incision in the thorax in the opposite case. In alternative, the main catheter 100 can be delivered to the implant position transapically, through an aperture created at the apex of the LV. When implant 10 is implanted transatrially, the main catheter 100 is inserted directly into the left atrium after thoracotomy.

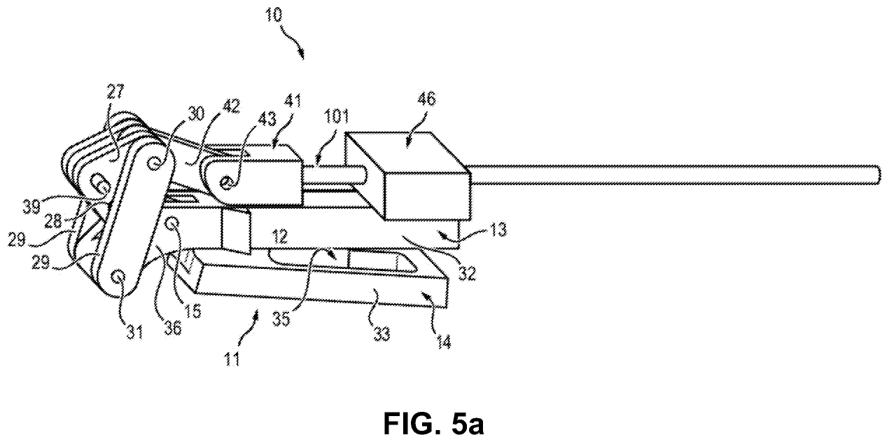

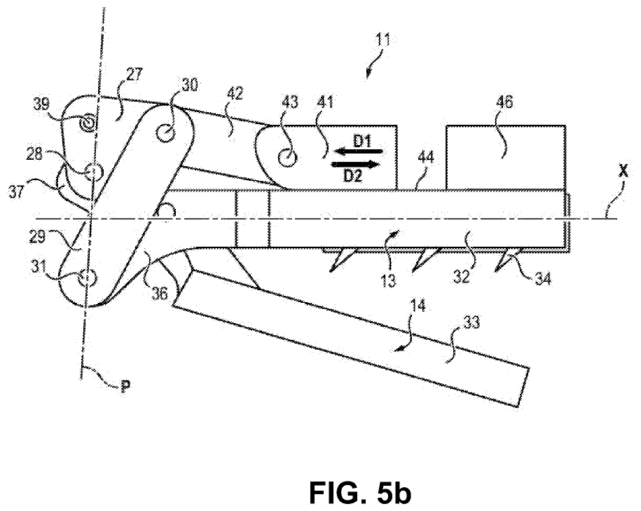

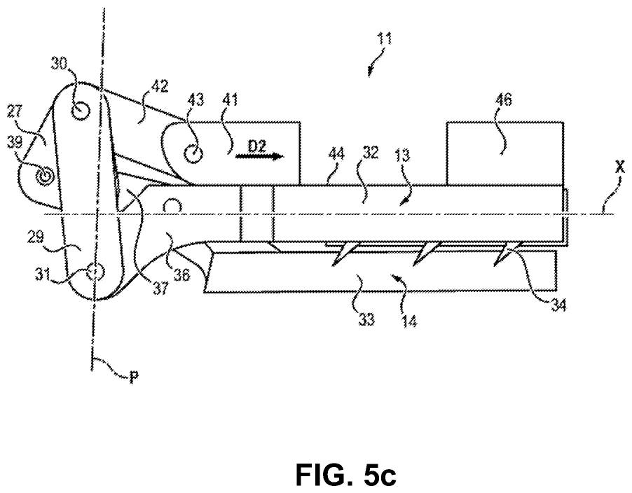

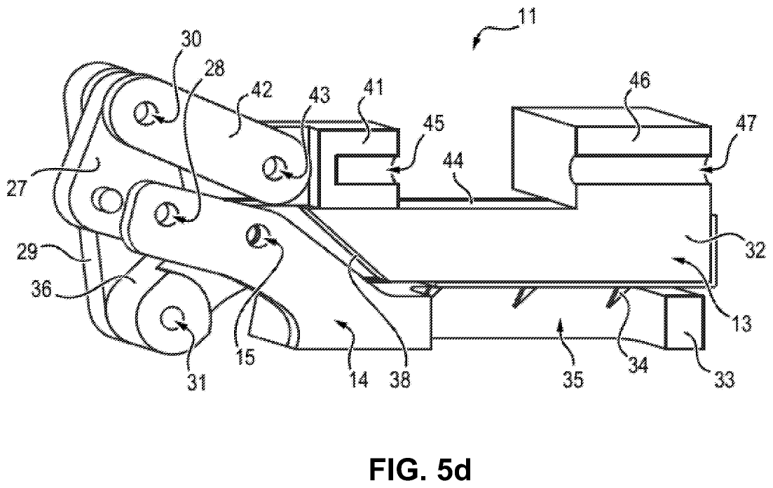

In reference with FIGS. 5a to 5c, the clamp 11 comprises a body 13 extending along a longitudinal axis X and a jaw 14 hinged for rotation relative to the body 13 about a first axis of rotation 15 perpendicular to the longitudinal axis X. The jaw 14 of the clamp 11 is configured to rotate about the first axis of rotation 15 relative to the body 13 between an open position and a closed position in which the body 13 and the jaw 14 of the clamp 11 are able to grip one of the leaflets (e.g. the posterior leaflet PL) of the mitral valve MV, thereby securing the implant 10 to the said leaflet.

Clamp 11 is coupled with the distal end of an actuating shaft 101 through which clamp 11 is opened and closed. For example, the shaft 101 is partially mounted inside a delivery catheter, with the distal end of the shaft 101 being arranged outside the delivery catheter. In the first configuration, the shaft 101 is attached to the delivery catheter and in the second configuration, the shaft 101 is free to slide along the delivery catheter. Thus, in the first configuration, the shaft 101 cannot slide inside the delivery catheter. The shaft 101 and the delivery catheter therefore move as one unit. In the second configuration, however, the shaft 101 can move back and forth by sliding inside the delivery catheter.

FIG. 5b shows clamp 11 in an intermediate position between the open and closed positions.

Clamp 11 includes a rotary drive 27 configured to rotate the jaw 14 relative to the body 13 about the first axis of rotation 15 between the open and closed positions.

For example, the rotary drive 27 includes a connecting rod which rotates with the jaw 14 about a second axis of rotation 28. The second axis of rotation 28 is parallel and not to be confused with the first axis of rotation 15. The movement of the connecting rod thus causes the jaw 14 to pivot relative to the body 13 about the first axis of rotation 15 between the open and closed position.

Clamp 11 can also include a locking device 27, 29 configured to lock clamp 11 in the closed position. Locking the clamp 11 in the closed position reduces the risk of unintentional opening of the clamp 11 once the implant 10 is attached to the posterior leaflet PL of the mitral valve MV.

FIG. 5c shows the clamp 11 in a closed and locked position.

For example, the clamp 11 comprises at least one rocker element 29 which is rotatably articulated with the connecting rod about a third axis of rotation 30 on the one hand and with the body 13 of the clamp 11 about a fourth axis of rotation 31 on the other hand. The third and fourth axes of rotation 30, 31 are parallel and distinct from the first and second axes of rotation 15, 28.

The rocker element 29 and the connecting rod 27 form a toggle lever. The toggle lever is configured to move between an unlocked configuration in which the third axis of rotation is located on a first side of the plane P formed by the second and fourth axes of rotation 28, 31, with the clamp 11 in the open position, and a locked configuration in which the third axis of rotation 30 is located on a second side of said plane P, with the clamp 11 in the closed position.

The clamp 11 comprises, for example, two rocker elements 29 arranged on either side of the body 13 and the connecting rod 27.

The body 13 and the jaw 14 each have a gripping part 32, 33. The gripping parts 32, 33 of the body 13 and the jaw 14 are arranged opposite each other and are configured to grip the posterior leaflet PL of the mitral valve MV, when clamp 11 is in the closed position.

The gripping part 32 of the body 13 accommodates the shutter 12. For this purpose, for example, the gripping part 32 of the body 13 is inserted into groove 19 in the first side of the balloon 12.

In addition, the gripping part 32 of the body 13 may have a number of spikes 34 which are configured to fit into the posterior leaflet PL of the mitral valve MV. The spikes 34 thus improve the grip of the clamp 11 on the PL posterior leaflet of the mitral valve MV.

For example, the spikes 34 extend generally perpendicularly from one side of the gripping part 32 of the body 13 to the jaw 14. For this purpose, a plate with the points 34 is for example attached to the side of gripping part 32 of the body 13 opposite the jaw 14.

Preferably, one or more through-openings 35 are made in the gripping part 33 of the jaw 14, opposite the points 34 of the body 13. The opening(s) 35 is (are) configured to receive the tips 34 of the body 13, when the clamp 11 is in the closed position. This ensures that the closing of clamp 11 is not hindered by the tips 34 of the body 13. The opening(s) 35 can be replaced by one or more receptacles.

Alternatively, the pins 34 are supported by the gripping part 33 of the jaw 14, e.g. via the plate, and if necessary, the opening(s) 35 is (are) made in gripping part 32 of the body 13.

The body 13 and the jaw 14 each have an actuating part 36, 37 with the first and fourth rotational axes 15, 31 and the first and second rotational axes 15, 28 respectively. The actuating parts 36, 37 of the body 13 and the jaw 14 are arranged opposite each other. For example, the first axis of rotation 15 is located on the actuating part 36 of the body 13 between the gripping part 32 of the body 13 and the fourth axis of rotation 31.

The first rotational axis 15 is, for example, located on the actuating part 37 of the jaw 14 between the gripping part 33 of said jaw 14 and the second rotational axis 28.

The second axis of rotation 28 is, for example, positioned on an opposite side of the gripping part 33 of the jaw 14, while the fourth axis of rotation 31 is positioned on an opposite side of the gripping part 32 of the body 13.

For this purpose, for example, a through-opening 38 is provided in actuating part 36 of the body 13 to accommodate the actuating part 37 of the jaw 14 and to allow the second pivot pin 28 to be located on a side opposite to gripping part 33 of the jaw 14. The body 13 and the jaw 14 thus intersect at the first axis of rotation 15.

The clamp 11 can also include a blocking device 39 configured to lock the toggle lever 27, 29 in a locked configuration. Locking the clamp 11 in the closed and locked position further reduces the risk of unintentional opening of the clamp 11 once the implant 10 is attached to the posterior leaflet PL of the mitral valve MV.

For this purpose, the connecting rod 27 includes, for example, a stop 39 configured to come into contact with the rocker element(s) 29 in the locked configuration of the toggle lever. For example, stop 39 is formed by an axis parallel to the second and third axes of rotation 28, 30. An example of clamp 11 with stop 39 is shown in FIGS. 5b to 5c.

Alternatively, jaw 14 has a protrusion 40 extending from actuating part 37 of jaw 14 and configured to contact connecting rod 27 in the locked configuration of the knee joint.

Clamp 11 may also include a jaw shifter 41, 42 configured to move the connecting rod 27 and thereby cause the jaw 14 to pivot relative to the body 13 about the first axis of rotation 15 between the open and closed position.

The displacement device 41, 42 comprises, for example, a pusher 41 configured to move translationally along the longitudinal axis X relative to the body 13 of the clamp 11, and a lever element 42 articulated rotationally relative to the connecting rod 27 about the third axis of rotation 30 on the one hand and relative to the pusher 41 about a fifth axis of rotation 43 on the other hand. The fifth axis of rotation 43 is parallel and not to be confused with the first, second, third and fourth axes of rotation 15, 28, 30, 31.

Thus, when the pusher 41 moves in translation along the longitudinal axis X relative to the body 13, it causes the third axis of rotation 30 to move via the lever element 42. When the pusher 41 is moved in a first direction D1, the jaw 14 pivots about the first axis of rotation to the clamped position PS and the third axis of rotation 30 is driven to the locked configuration. When the pusher 41 is moved in a second direction D2, opposite to the first direction D1, the jaw 14 pivots about the first axis of rotation 15 to the spread position and the third axis of rotation 30 moves away from the locked configuration.

Preferably, the pusher 41 is configured to move translationally along the longitudinal axis X by sliding against a face 44 of the body 13, opposite the gripping part 33 of the jaw 14. In this way, the movement of the pusher 41 is guided by the body 13.

For example, the pusher 41 is provided with an opening 45 extending along the longitudinal axis X. The opening 45 is designed to cooperate with the distal end of the shaft 101. This allows the operator to manipulate the proximal end of the shaft 101 to move the pusher 41 and thus activate the opening and closing of the clamp 11, and possibly its locking.

In addition, the body 13 can be fitted with a guide element 46 which is configured to guide the translation of the distal end of the shaft 101 along the longitudinal axis X.

The guide element 46 extends, for example, from the side 44 of the body 13 opposite the grip portion 33 of the jaw 24 and includes a through-opening 47 extending along the longitudinal axis X and configured to slidably receive the distal end of the shaft 101. The opening 47 of the guide element 46 and the opening 45 of the pusher element 40 are coaxial and arranged opposite each other. The opening 47 thus guides the distal end of the shaft 101 in a translational movement along the longitudinal axis X, which can then move the pusher 41 in a translational movement along the longitudinal axis X and thus open and close the clamp 11.

The clamp 11, for example, is covered with a Dacron® coating. This material promotes the endothelialisation (colonisation of tissue by endothelial cells) of the implant, which improves its hold on the implant site and reduces the risk of thrombus formation (blockage of a blood vessel). In other words, the clamp 11, for example, is made of titanium and is covered with a thin layer of Dacron® coating. Alternatively, the clamp 11 is made of a chromium-cobalt alloy or any other suitable biocompatible and/or haemocompatible material.

Now turning to FIGS. 6a and 6b, the shutter 12 comprises a meshed structure 50 adapted to pass elastically from a contracted configuration to an expanded configuration. In the expanded configuration, the shutter 12 is able to fill at least partially the opening portion O1 remaining between the leaflets AL, PL during systole, when the implant is clamped to the first leaflet, so as to limit a backflow of blood through the opening portion O1 during systole.

When the implant 10 is moved along the main catheter 100 to the mitral valve MV, the meshed structure 50 is kept in the contracted configuration by the catheter 100. Once the implant 10 exits the catheter 100, the meshed structure 50 passes automatically from the contracted configuration to the extended configuration. In use, the meshed structure remains in the expanded configuration.

The meshed structure 50 of the shutter 12 comprises interconnected wires 52. The wires 52 move related to each other when the configuration of the meshed structure 50 changes. The meshed structure 50 has a first end 54 and a second end 56 opposite to the first end.

The first end 54 of the meshed structure is affixed to the body 13.

In the contracted configuration, the meshed structure 50 extends along the longitudinal axis X and is unbent.

When the meshed structure 50 passes from the contract configuration to the expanded configuration, the wires 52 naturally bend towards the clamp 11 and move away from each other, thereby causing the second end 56 to enlarge.

In the expanded configuration, the meshed structure 50 is curved in two directions perpendicular to each other. It defines an open cavity facing the pusher 41. Moreover, the second end 56 of the meshed structure 50 extends about the first end 54 thereof. In the expanded configuration, the dimensions of the meshed structure measured in a plane perpendicular to longitudinal axis X are larger than in the contracted configuration.

The meshed structure of shutter 12 is preferably made of a biocompatible and/or haemocompatible material. Indeed, the biocompatible and haemocompatible aspect of shutter 12 is advantageous for the implantation of the device in the patient's heart. A non-haemocompatible material, for example, would cause blood coagulation around the device and a vital risk for the patient. Likewise, the meshed structure of shutter 12 is advantageously able to bend to fit into a catheter, but also be able to unfold, once the implant 10 is attached to the leaflet, at the heart valve, while maintaining its mechanical properties throughout the insertion of the device into the heart. A material such as Nitinol (a known Nickel/Titanium alloy), for example, would enable these functions to be fulfilled.

The meshed structure 50 as such is able to limit a backflow of blood through the opening portion during systole, once the implant 10 is installed in a heart. The denser the meshed structure 50, the more the implant 10 prevents blood from flowing backwards.

Advantageously, the shutter 12 further comprises a membrane 58 at least partially covering the meshed structure 50. This membrane 58 further contributes to fill the portion O1 of the opening O, thus improves the effect of limiting the backflow of blood.

The membrane 58 may be made of a biocompatible and/or haemocompatible material. In an embodiment, the membrane is made of a synthetic organic polymer, such as PTFE (polytetrafluoroethylene). This material has a glass transition temperature of −30° ° C. and a melting temperature of 327° C. Thus, with a body temperature of approximately 37° C., PTFE is a suitable material for providing rigidity without being brittle once inserted into a patient's heart. It is also hydrophobic and resists repeated bending and folding.

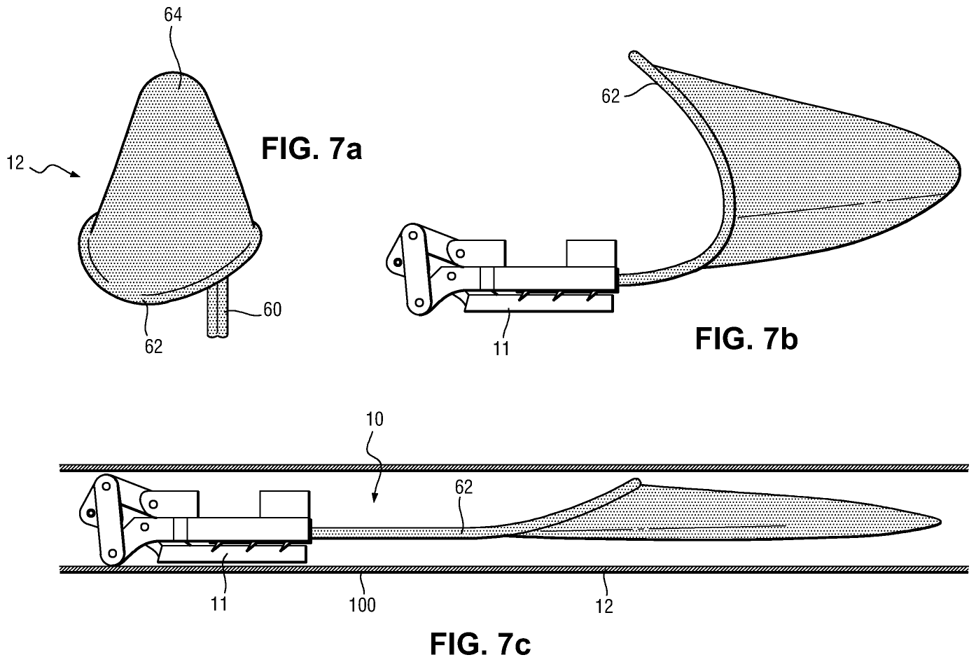

FIGS. 7a-7c shows a second embodiment of shutter 12. This second embodiment of the shutter 12 differs from the first embodiment shown in FIG. 6a by the following aspects. The second embodiment of the shutter 12 comprises a wire 62 and a membrane 64 curved in two directions perpendicular to each other. Wire 62 forms a loop. Membrane 64 is attached onto wire 62. The wire 62 is connected to the anchoring structure 60. When in the contracted configuration, the loop formed by wire 62 is unbent. When in the expanded configuration, the membrane 64 kept open by the wire 62 defines an open cavity. The loop formed by wire 62 is bent, and wire 62 defines an access of the open cavity. When in the contracted configuration, the wire 62 still defines the open cavity, but this cavity has a smaller volume.

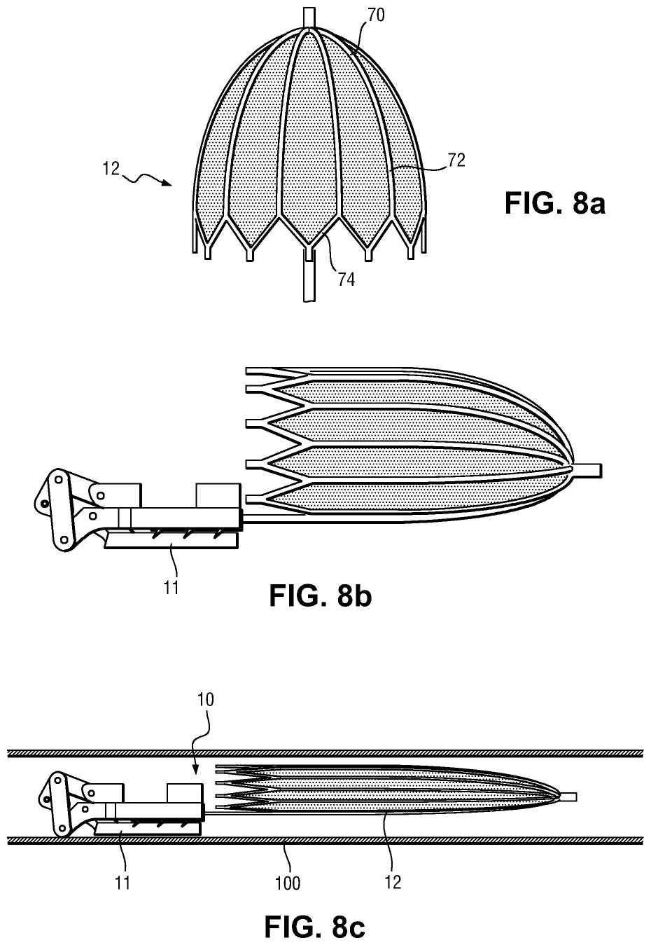

FIGS. 8a-8c show a third embodiment of shutter 12. This third embodiment of the shutter 12 differs from the first and second embodiment by a meshed structure 70 with wires arranged in a further manner. The meshed structure 70 comprises longitudinal wires 72 which extend about the open cavity in the expanded configuration. The meshed structure 72 further comprise transversal wires 74 which interconnect the longitudinal wires 72. Each transversal wire 74 is rotatably mounted to two adjacent longitudinal wires 72. The transversal wires 74 form together a zig zag shape defining an access to the open cavity. When in the contracted configuration, the meshed structure 70 still defines the open cavity, but this cavity has a smaller volume, like in the second embodiment.

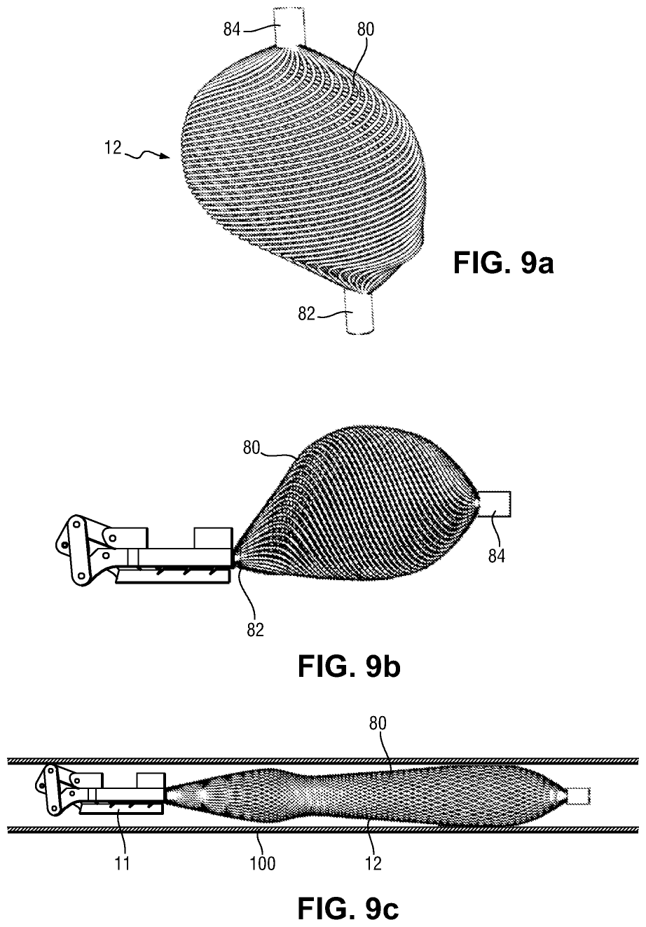

FIGS. 9a-9c show a fourth embodiment of shutter 12. This fourth embodiment of the shutter 12 differs from the first and second embodiment by a meshed structure 80 with wires arranged in still another manner. The meshed structure 80 has two opposite ends 82, 84. The wires of the meshed structure extend between ends 82, 84. The wires actually define together a closed cavity which increases in volume when ends 82, 84 are moved toward each other in a direction parallel to longitudinal axis X, and which decreases in volume when said ends 82, 54 are moved away from each other in this direction.

End 82 is affixed to the body 13 of the clamp 11. In the contracted configuration, the ends 82, 84 extend along the longitudinal axis X. When the meshed structure passes from the contracted configuration to the expanded configuration, the end 82, 84 naturally move towards each other, which causes the wires to bend and the closed cavity to increase in volume. Moreover, the ends 82 and 84 may further move so as to become off-axis.

A method of placing the implant 10 by a transseptal route comprises the steps described below.

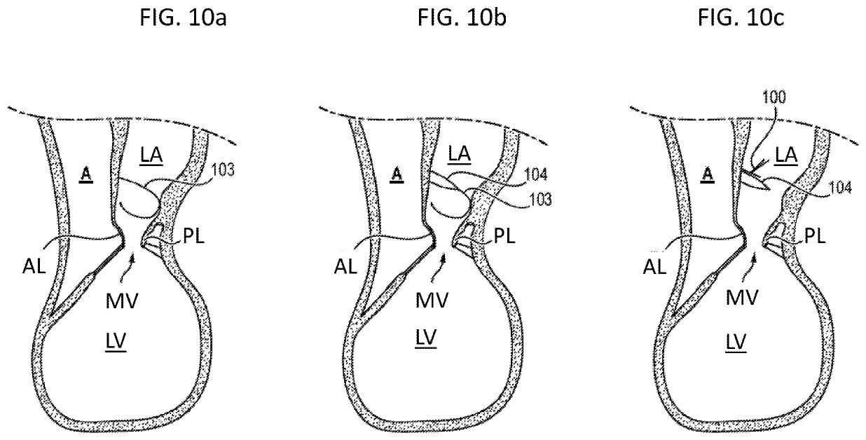

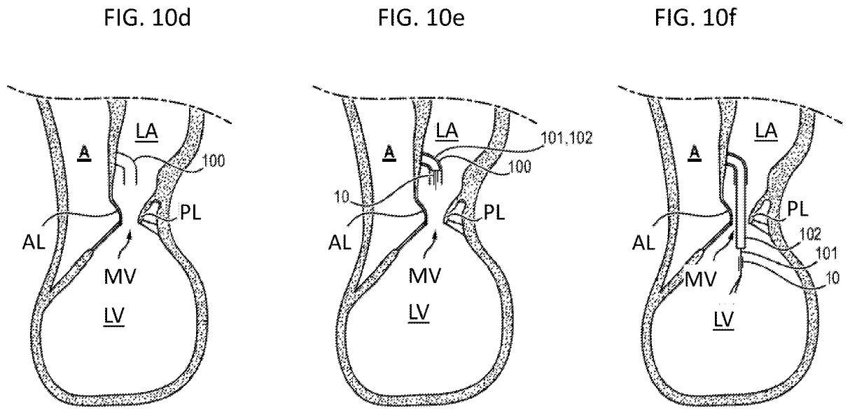

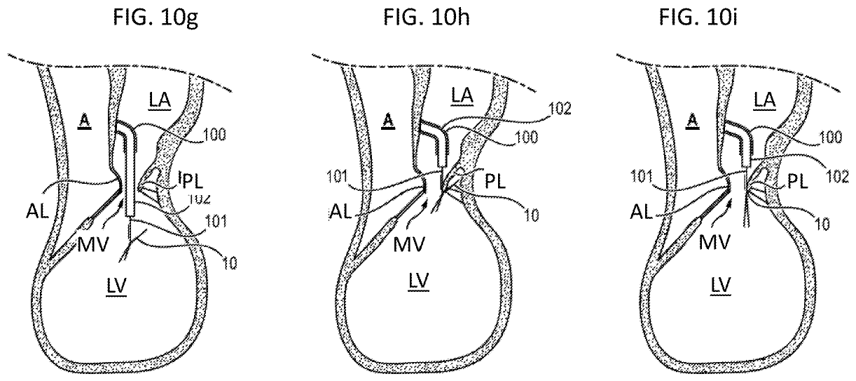

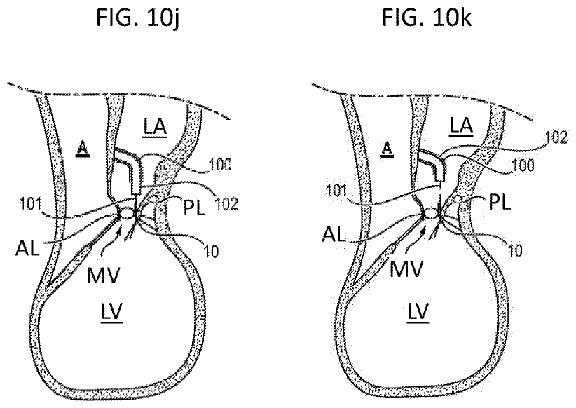

First, a guide is inserted into the femoral vein, up to the right atrium RA and then a transseptal catheterization is performed (through the inter-atrial septum) up to the left atrium LA and the mitral ring MR, as shown in FIG. 10a.

Then, a dilator 104 is threaded onto guide 103, which in turn is inserted up to the left atrium LA. The dilator 104 is then guided through the guide 103 to the left atrium LA. The dilator 104 dilates the tissues of the inter-atrial septum to allow the insertion of the main catheter 100, as shown in FIG. 10b.

Then the guide catheter 103 is removed.

The main catheter 100 is in turn inserted over the guidewire through the inter-atrial septum to the left atrium LA. To do this, the main catheter 100 is, for example, threaded around the dilator 104, as shown in FIG. 10c.

Then the dilator 104 is removed.

The distal end 105 of the main catheter 100 is oriented so that it extends generally coaxially with the mitral ring MR. The distal end 105 of the main catheter 100 thus includes an adjustable part, as shown in FIG. 10d.

The shaft 101, whose distal end cooperates with the implant 10, and possibly the delivery catheter 102, are introduced into the main catheter 100. The shaft 101 and the delivery catheter 102 are then in the first configuration in which the shaft 101 and the delivery catheter 102 move as one unit. The clamp 11 is in the closed position. The shutter device 12 is in the contracted configuration, as schematically shown in FIG. 10e.

Then the distal end of the shaft 101 and, if applicable, the distal end of the delivery catheter 102 exit the main catheter 100 and pass through the mitral valve MV until the distal end of the delivery catheter 102 is positioned in the left ventricle LV. The shutter 12 of the implant 10 is then oriented towards the anterior leaflet AL of the mitral valve MV, while the jaw 14 of the clamp 11 is oriented towards the posterior leaflet PL of the mitral valve MV, as shown in FIG. 10f. Clamp 11 is still in the closed position. The shutter 12 is still in the contracted configuration.

Then, the clamp 11 is opened. To do this, the shaft 101 pulls pusher 41 towards the end of the body 13. The shaft 101 and the wire guide 102 are then in the second configuration in which the shaft 101 is free to slide inside the wire guide 102. This step is shown in FIG. 10g.

Then, the shaft 101 and possibly the delivery catheter 102 move towards the mitral valve VM so that the posterior leaflet PL of the mitral valve MV is positioned between the body 13 and the jaw 14 of the still open clamp 11. The shaft 101 and the delivery catheter 102 are then in the first configuration in which the shaft 101 and the delivery catheter 102 move in one piece. The step is shown in FIG. 10h.

The clamp 11 is closed and possibly locked. The implant 10 is thus fixed to the posterior leaflet PL of the mitral valve MV. To do this, the shaft 101 pushes the pusher 41 downstream towards the apex of the body 13 of implant 10 opposite the obturator 12. The shaft 101 and the delivery catheter 102 are then in the second configuration in which the shaft 101 is free to slide inside the delivery catheter 102. This step is shown in FIG. 10i.

Besides, the meshed structure of shutter 12 passes from the contracted configuration to the expanded configuration. This step is shown in FIG. 10j.

Then, implant 10 is released. For this purpose, shaft 101 is, for example, decoupled from pusher 41. This step is shown in FIG. 10k. Then, the shaft 101, possibly the delivery catheter 102 and the main catheter 100 are removed from the patient's body.

All these steps can be carried out under cardiac fluoroscopic control and ETO (trans-esophageal echocardiography) control.

The method described above is particularly advantageous as it offers reduced risks for the patient compared to open-heart surgery.

The implant 10 and the method described above are not only intended for placement on a mitral valve. The proposed implant can also be placed on heart valves other than the mitral valve, in particular the other atrioventricular valve, i.e. the tricuspid valve.

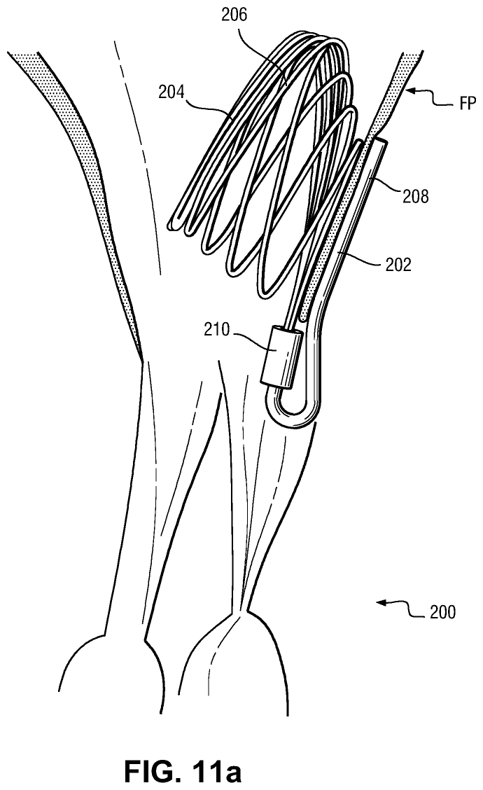

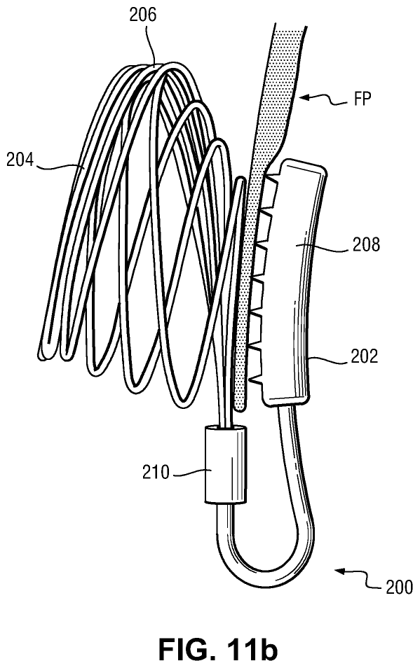

A second implant 200 according to a second embodiment is depicted in FIGS. 11a and 11b. This implant 200 is an alternative to implant 10.

Like the implant 10 described above, the second implant 200 comprises:
a clamp 202 movable between an open position and a closed position in which the clamp is able to grip a first leaflet among the leaflets of the heart valve (for instance the posterior leaflet PL), so as to fix the implant on the first leaflet,
a shutter 204 comprising a meshed structure 206 adapted to pass automatically from a contracted configuration to an expanded configuration in which the shutter is able to fill at least partially an opening portion remaining between the two leaflets during a systole, when the implant is attached to the first leaflet, so as to limit a backflow of blood through the opening portion during the systole.

The shutter 204 may be identical to the shutter 12 of the implant described above. It may in particular include a membrane (not illustrated) at least partially covering the meshed structure 206.

The clamp 202 of the second implant 200 comprises a fastening arm 208 capable of facing a portion of the meshed structure in the closed position. Thus, a leaflet of the heart valve can be enclosed by a first bearing surface defined by the fastening arm and a second bearing surface of the meshed structure 204. Put differently, the meshed structure not only contributes to fill at least partially the opening portion O1 remaining between the two leaflets of the heart valve, but also contributes to form the clamp 202, thus to fix the second implant 200 on the first leaflet.

The fastening arm 208 is bendable relative to the meshed structure 206 about an axis of rotation. The clamp 202 can be set in the open position by bending the fastening arm 208 about the axis of rotation, such that the fastening arm is moved away from the meshed structure 206 (in other words, by moving the first bearing surface from the second bearing surface).

The fastening arm 208 is made of a resilient material which naturally returns by bending into the closed position. This material may be for instance Nitinol.

The fastening arm 208 has a first end portion that actually defines the first bearing surface. Preferably, the fastening arm is serrated (as shown in FIG. 11b) or corrugated, such that the implant is more efficiently secured to the target leaflet of the heart valve.

The fastening arm 208 further comprises a second end portion, and an intermediate portion interconnecting the first and second end portions.

The second implant 200 further comprises a sleeve 210 which interconnects the meshed structure 206 and the fastening arm.

The sleeve 210 is tubular. The sleeve 210 defines an inner cavity, a first opening and a second opening opposite to the first opening relative to the inner cavity. Both openings give access to the inner cavity. Both openings are coaxial.

End portions of wires of the meshed structure are enclosed in the sleeve 210, such that the sleeve 210 keeps said end portions fixed relative to each other. The wires of the meshed structure protrude out of the sleeve through the first opening.

Similarly, the second end portion of the fastening arm 208 in enclosed in the sleeve 210, such that the sleeve keeps said end portion fixed relative to the end portions of the wires. The fastening arm 208 protrudes out of the sleeve through the second opening.

When the clamp 202 is in the closed position, the intermediate portion of the fastening arm 208 is bent on itself so as to face the sleeve 210, thereby allowing the first end portion thereof to face the meshed structure 206 protruding out of the sleeve through the opposite (first) opening.

The open position of the clamp 202 may be a position wherein the intermediate portion is unbent and made substantially rectilinear.

The second implant 200 may be inserted in a delivery catheter while the intermediate portion of the fastening arm is in this unbent position and while the meshed structure 206 is in its contracted configuration. An inner surface of the catheter prevents the fastening arm to come back naturally in its closed position. Once the second implant 200 is extracted from said catheter, the fastening arm naturally bends such that the clamp 202 reaches the closed position, and the meshed 206 structure naturally expands, i.e. switches from its contracted configuration to its expanded configuration.

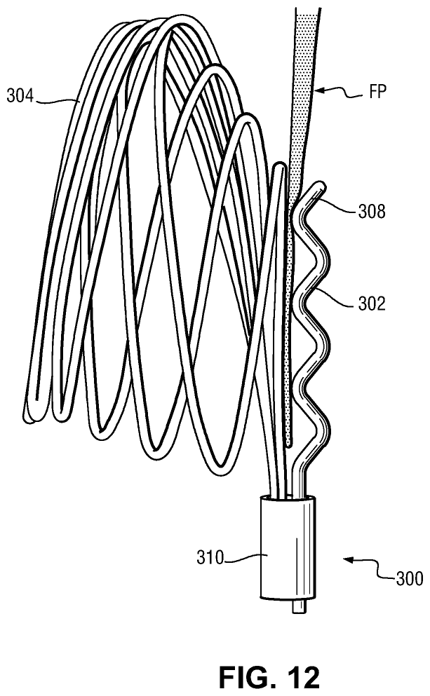

A third implant 300 according to a third embodiment is depicted in FIG. 12. The third implant comprises a clamp 302 comprising a fastening arm 308, and a shutter 304 similar to shutter 204. The third implant differs from the second implant 200 in that the fastening arm 308 protrudes out of the sleeve through the first opening. Unlike the second implant 200, the intermediate portion of the fastening arm 308 is maintained in a bent configuration in a catheter and unbends naturally once it is extracted from the catheter.

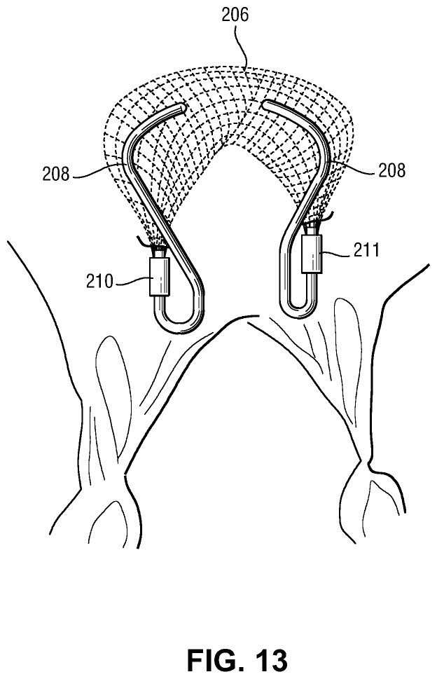

The second implant 200 or the third implant 300 may comprise more than one fastening arm 208, 308, so as to define multiple bearing surface. For example, the embodiment shown in FIG. 13 comprises two fastening 208, 209 arms and two sleeves 210, 211. Each fastening arm protrudes from one on the sleeves. As a result, a leaflet of the heart valve can be enclosed by three bearing surfaces, which are respectively defined by the meshed structure, the first fastening arm 208 and the second fastening arm 209. Each sleeve 210, 21 encloses end portions of a subset of wires of the meshed structure 206. Of course, the number of fastening arms and sleeves may be greater than two in other embodiments. Having more than one fastening arm is advantageous to stabilize the implant relative to the first leaflet. Indeed, if the second implant 200 or the third implant 300 comprise one single arm which is substantially linear along an axis, the implant may be prone to tilt about said axis.

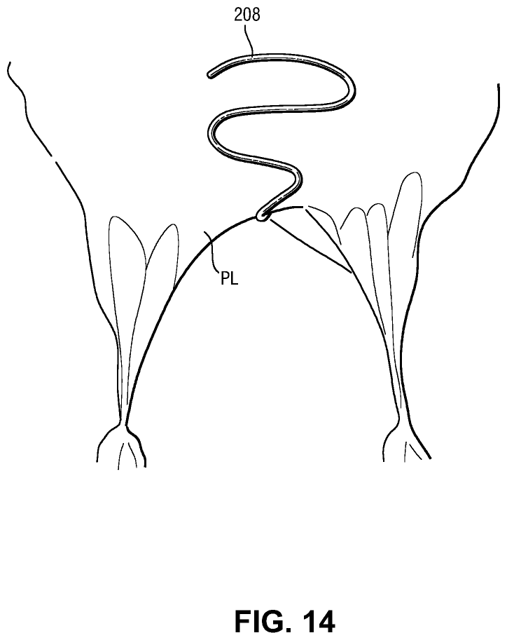

Improving the stability of the second implant 200 or the third implant 300 can also be achieved by causing the fastening arm 208 or 308 to have a zig zag shape in a plane parallel to the axis of rotation of said fastening arm relative to the meshed structure, as shown in FIG. 14.

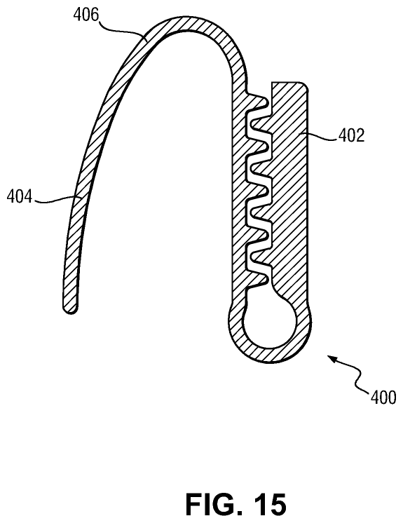

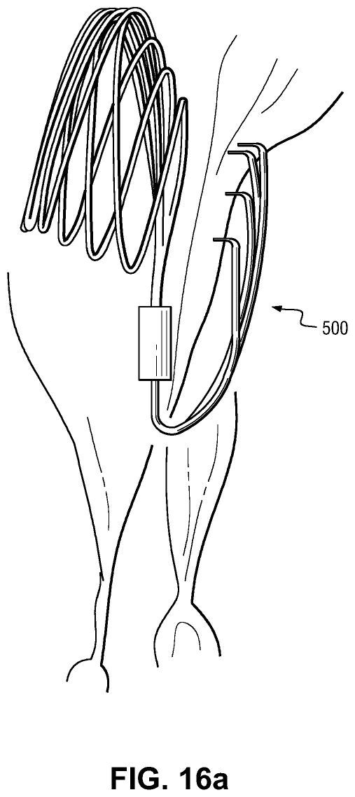

A fourth implant 400 according to a fourth embodiment is depicted in FIG. 15. The fourth implant comprise a clamp 402 and a shutter 404 comprising a meshed structure 406 playing the same functions as the clamps and shutters of the implants described above. However, unlike the first, second and third implants 10, 20, 300 described above, the meshed structure and the clamp of the fourth implant 400 are parts of a single piece. This single piece is preferably made of Nitinol.

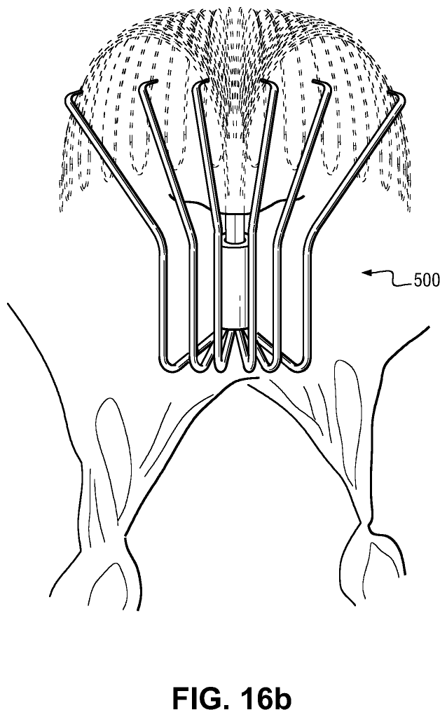

A fifth implant 500 according to a fifth embodiment is depicted in FIGS. 16a, 16b. The fifth implant 500 is similar to the second implant 200, except that it comprises many fastening arms, for instance six. All fastening arms have respective second end portions enclosed in the same sleeve. When the clamp is set in the closed position, all fastening arms spread so as to contact different zones of a leaflet. This is an alternate configuration improving the stability of the clamping.

1. An implant for heart valve repair, the implant comprising:
a clamp movable between an open position and a closed position in which the clamp is able to grasp a leaflet of a heart valve, so as to fix the implant on the leaflet, and
a shutter comprising a structure adapted to pass automatically from a contracted configuration to an expanded configuration in which the shutter is able to fill at least partially an opening portion remaining between the leaflet and at least one further leaflet of the heart valve, when the implant is attached to one leaflet, so as to limit a backflow of blood through the opening portion when the heart valve closes, and wherein the structure is curved in two directions perpendicular to each other in the expanded configuration.

2. The implant of claim 1, wherein the structure is a metallic mesh.

3. The implant of claim 1, wherein the structure is made of a polymer selected from the group consisting of polyolefin, vinyl polymer, saturated polyester, polyurethane or any other biocompatible polymer.

4. The implant of claim 1, wherein the shutter comprises a membrane at least partially covering the structure.

5. The implant of claim 1, wherein:
the shutter defines a first bearing surface, and
the clamp comprises a fastening arm defining a second bearing surface capable of facing the first bearing surface when the clamp is in the closed position, such that the first bearing surface and the second bearing surface enclose the leaflet.

6. The implant of claim 1, wherein at least one of the first bearing surface and the second bearing surface is serrated or corrugated.

7. The implant of claim 5, wherein the fastening arm is rotatable about an axis of rotation relative to the structure and has a zig zag shape.

8. The implant of claim 4, wherein the clamp comprises at least one further fastening arm defining a third bearing surface capable of facing the first bearing surface when the clamp is in the closed position, such that the first bearing surface, the second bearing surface and the third bearing surface enclose the first leaflet therebetween.

9. The implant of claim 1, wherein the shutter comprises wires which are free to move relative to each other when the meshed structure passes from the contracted configuration to the expanded configuration, the implant further comprising a sleeve enclosing end portions of the wires.

10. The implant of claim 9, wherein the clamp comprises a fastening arm, and the sleeve encloses an end portion the fastening arm.

11. The implant of claim 9, wherein the sleeve has an opening, the wires and the fastening arm protruding out of the sleeve through the opening.

12. The implant of claim 9, wherein the sleeve has a first opening and a second opening opposite the first opening, the wires protrude out of the sleeve through the first opening and the fastening arm protrudes out of the sleeve through the second opening.

13. The implant of claim 1, wherein the structure and the clamp are parts of a single piece.
